(19) United States
(12) Patent Application Publication
(10) Pub. No.: US 2025/0166132 A1
(43) Pub. Date: May 22, 2025

(54) APPARATUS AND METHOD FOR DENOISING OF MEDICAL IMAGE

(71) Applicant: CLARIPI INC., Seoul (KR)

(72) Inventors: Jong Hyo KIM, Seoul (KR); Tae Jin KIM, Seoul (KR); Chul Kyun AHN, Seoul (KR)

(21) Appl. No.: 18/811,465

(22) Filed: Aug. 21, 2024

(30) Foreign Application Priority Data
Nov. 16, 2023 (KR) 10-2023-0159232

(51) Int. Cl. G06T 5/50; G06T 5/70; G16H 30/40

(52) U.S. Cl. CPC G06T 5/50; G06T 5/70; G16H 30/40; G06T 2207/20221; G06T 2207/20224

(57) ABSTRACT

Disclosed is an apparatus for denoising a medical image, including: an image processing module configured to extract a noise component from a medical image for processing by inputting the medical image for the processing to a noise extraction deep learning model trained in advance, and generate a noise-removed image by subtracting the noise component from the medical image for the processing, wherein the noise extraction deep learning model is trained using a simulation noise component image generated by a noise simulator and a simulation low-quality image generated based on the simulation noise component image as a pair.

### CROSS REFERENCE TO RELATED APPLICATION

Priority to Korean patent application number 10-2023-0159232 filed on Nov. 16, 2023 the entire disclosure of which is incorporated by reference herein, is claimed.

### BACKGROUND OF THE INVENTION

### Field of the Invention

The disclosure relates to an apparatus and method for denoising a medical image, and more particularly to an apparatus and method for denoising a medical image to remove noise from the medical image.

### Description of the Related Art

In general, an X-ray, a computed tomography (CT), a magnetic resonance imaging (MRI), and the like medical apparatuses are used to acquire medical images. In modern medicine, the medical images acquired through such medical apparatuses are used as a very important basis for the presence and characteristics of lesions to make decisions in a process of diagnosing and treating a patient.

A related art of denoising a medical image has already been disclosed in Korean Patent Publication No. 2014-0134903 (titled “METHOD AND APPARATUS FOR IMPROVING QUALITY OF MEDICAL IMAGE,” and published on Nov. 25, 2014). This related art is to improve the quality of a medical image by removing noise from the medical image.

With the advancement of artificial intelligence (AI) technology, various technologies of using a deep learning model trained in advance to reduce noise have been researched and developed. However, a large amount of data is required to train the deep learning model. In particular, it is required to sufficiently secure medical images of various qualities in training the deep learning model for denoising. However, the number of medical images for training the deep learning model is very limited, and thus a noise reduction technology for the medical images based on noise simulation training has been required.

### SUMMARY OF THE INVENTION

An aspect of the disclosure is to provide an apparatus and method for denoising a medical image, in which a larger number of medical image sets for training is generated based on a small number of medical image sets by a noise simulator, and a deep learning model for noise extraction is trained based on the generated medical image sets, thereby denoising the medical images effectively.

According to the disclosure, an apparatus for denoising a medical image includes: an image processing module configured to extract a noise component from a medical image for processing by inputting the medical image for the processing to a noise extraction deep learning model trained in advance, and generate a noise-removed image by subtracting the noise component from the medical image for the processing, wherein the noise extraction deep learning model is trained using a simulation noise component image generated by a noise simulator and a simulation low-quality image generated based on the simulation noise component image as a pair.

The simulation low-quality image may be generated by combining the simulation noise component image and a normal-quality medical image.

According to an embodiment of the disclosure, the noise simulator may be configured to generate the simulation noise component image by inputting a set of the normal-quality medical images, of which a domain is converted into a sinogram domain, to a noise generation model trained in advance.

The noise generation model may be provided as a model, of which parameters are varied depending on training, and repeatedly trained to minimize a loss due to difference between a noise component image for training and the generated simulation noise component image.

The generated simulation noise component image may be generated by inputting a set of normal-quality medical images for training, of which a domain is converted into a sinogram domain, to the noise generation model.

According to another embodiment of the disclosure, the noise simulator may be configured to generate the simulation noise component image by inputting a set of the normal-quality medical images to a generative adversarial model trained in advance.

The generative adversarial model may be trained using the set of medical images for training, in which the normal-quality medical image and the noise component image are included forming a pair.

The generative adversarial model may be repeatedly trained to minimize a loss due to difference between a noise component image for training and a simulation noise component image generated by inputting a set of normal-quality medical images for training to the generative adversarial model.

According to still another embodiment of the disclosure, the noise simulator may be configured to generate a set of low-quality medical images by inputting a set of normal-quality medical images to a generative adversarial model trained in advance, and generate the simulation noise component image by subtracting the set of low-quality medical images from the set of normal-quality medical images.

The generative adversarial model may be trained using the set of medical images for training, in which the normal-quality medical image and the low-quality medical image are included forming a pair.

The generative adversarial model may be repeatedly trained to minimize a loss due to difference between a low-quality medical image for training and a simulation low-quality medical image generated by inputting a set of normal-quality medical images for training to the generative adversarial model.

Meanwhile, according to the disclosure, a method of denoising a medical image includes: inputting a medical image for processing to a noise extraction deep learning model trained in advance; extracting a noise component from the medical image for the processing through the noise extraction deep learning model; and generating a noise-removed image by subtracting the noise component from the medical image for the processing, wherein the noise extraction deep learning model is trained using a simulation noise component image generated by a noise simulator and a simulation low-quality image generated based on the simulation noise component image as a pair.

The simulation low-quality image may be generated by combining the simulation noise component image and a normal-quality medical image.

According to an embodiment of the disclosure, wherein the noise simulator is configured to generate the simulation noise component image by inputting a set of the normal-quality medical images, of which a domain is converted into a sinogram domain, to a noise generation model trained in advance.

The noise generation model may be provided as a model, of which parameters are varied depending on training, and repeatedly trained to minimize a loss due to difference between a noise component image for training and the generated simulation noise component image.

According to another embodiment of the disclosure, the noise simulator may be configured to generate the simulation noise component image by inputting a set of the normal-quality medical images to a generative adversarial model trained in advance.

The generative adversarial model may be trained using the set of medical images for training, in which the normal-quality medical image and the noise component image are included forming a pair.

The generative adversarial model may be repeatedly trained to minimize a loss due to difference between a noise component image for training and a simulation noise component image generated by inputting a set of normal-quality medical images for training to the generative adversarial model.

According to still another embodiment of the disclosure, the noise simulator may be configured to generate a set of low-quality medical images by inputting a set of normal-quality medical images to a generative adversarial model trained in advance, and generate the simulation noise component image by subtracting the set of low-quality medical images from the set of normal-quality medical images.

The generative adversarial model may be trained using the set of medical images for training, in which the normal-quality medical image and the low-quality medical image are included forming a pair.

The generative adversarial model may be repeatedly trained to minimize a loss due to difference between a low-quality medical image for training and a simulation low-quality medical image generated by inputting a set of normal-quality medical images training to the generative adversarial model.

In an apparatus and method for denoising a medical image according to the disclosure, a noise simulator is trained with a relatively small amount of medical image data, and a larger amount of medical image data for training is generated based on the noise simulator to train a deep learning model for noise extraction, thereby implementing noise reduction performance more effectively.

The technical effects of the disclosure are not limited to the aforementioned effects, and other unmentioned technical effects may become apparent to those skilled in the art from the following description.

### BRIEF DESCRIPTION OF THE DRAWINGS

FIG. 1 is a conceptual view of schematically showing an apparatus for denoising the medical image according to an embodiment,

FIG. 2 is a conceptual view of generating a simulation noise component image based on a first noise simulator according to an embodiment,

FIG. 3 is a flowchart of generating the simulation noise component image based on the first noise simulator according to an embodiment,

FIG. 4 is a conceptual view of showing how to train a noise model loaded into the first noise simulator according to an embodiment,

FIG. 5 is a conceptual view of generating a simulation noise component image based on a second noise simulator according to an embodiment,

FIG. 6 is a flowchart of generating the simulation noise component image based on the second noise simulator according to an embodiment,

FIG. 7 is a conceptual view of showing how to train a generative adversarial model loaded into the second noise simulator according to an embodiment,

FIG. 8 is a conceptual view of generating a simulation noise component image based on a third noise simulator according to an embodiment,

FIG. 9 is a flowchart of generating the simulation noise component image based on the third noise simulator according to an embodiment,

FIG. 10 is a conceptual view of showing how to train a generative adversarial model loaded into the third noise simulator according to an embodiment,

FIG. 11 is a conceptual view of generating a simulation low-quality image from a simulation noise component image according to an embodiment, and

FIG. 12 is a conceptual view of training a noise extraction deep learning model according to an embodiment.

### DETAILED DESCRIPTION OF THE INVENTION

Hereinafter, embodiments of the disclosure will be described in detail with reference to the accompanying drawings. However, the disclosure is not limited to the embodiments set forth herein, but may be implemented in various different ways. The embodiments are provided to only complete the disclosure and allow those skilled in the art to understand the category of the disclosure. In the accompanying drawings, the shape, etc. of an element may be exaggerated for clear description, and like numerals refer to like elements.

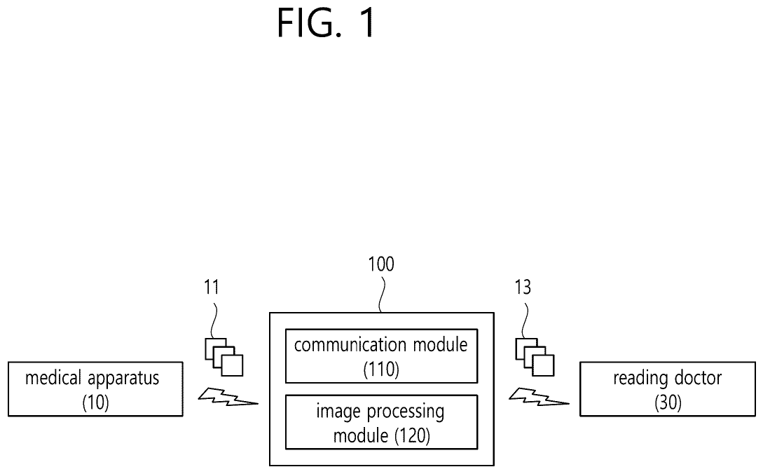

FIG. 1 is a conceptual view of schematically showing an apparatus for denoising the medical image according to an embodiment,

As shown in FIG. 1, an apparatus 100 for denoising a medical image according to an embodiment (hereinafter referred to as a noise reduction apparatus) may reduce noise components of a medical image 11 for processing, which is acquired from a medical apparatus 10, and transmit a readout image 13 with the reduced noise components to a reading doctor 30.

Here, the medical apparatus 10 may include a computed tomography (CT) apparatus, a magnetic resonance imaging (MRI) apparatus, and a positron emission tomography (PET) apparatus, etc., but there are no limits to the types of medical apparatus 10 and the formats of medical image acquired from the medical apparatus 10.

The noise reduction apparatus 100 may include a communication module 110 and an image processing module 120. The communication module 110 and the image processing module 120 may be provided as elements independent of each other, or provided together in a single computer system.

First, the communication module 110 may receive the medical images 11 for the processing from the medical apparatus 10, a server (not shown) or the like external apparatus. Further, the image processing module 120 inputs the medical images 11 for the processing to a noise extraction deep learning model 200 trained in advance, thereby extracting noise components from the medical image 11 for the processing. In addition, the image processing module 120 may generate a readout image 13 by subtracting the noise components from the medical image 11 for the processing.

Here, the noise extraction deep learning model 200 is trained using a simulation noise component image and a simulation low-quality image generated based on the simulation noise component image as a pair. Thus, the noise extraction deep learning model 200 may implement a function of extracting the noise components from the medical image 11 for the processing provided by the medical apparatus 10.

The simulation noise component images may be generated by noise simulators 310, 320, and 330 trained in advance. The noise simulators 310, 320, and 330 may selectively use a first noise simulator 310, a second noise simulator 320, and a third noise simulator 330 to generate the simulation noise component image.

Below, the first noise simulator 310, the second noise simulator 320, and the third noise simulator 330 according to an embodiment will be described in detail with reference to the accompanying drawings.

### <First Noise Simulator>

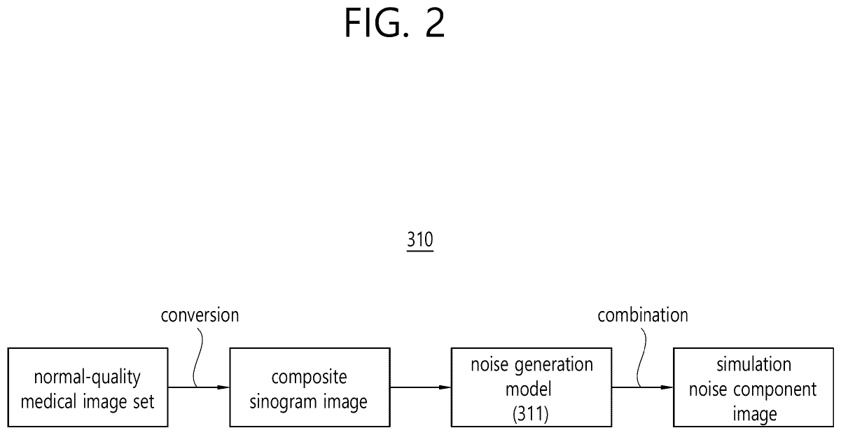

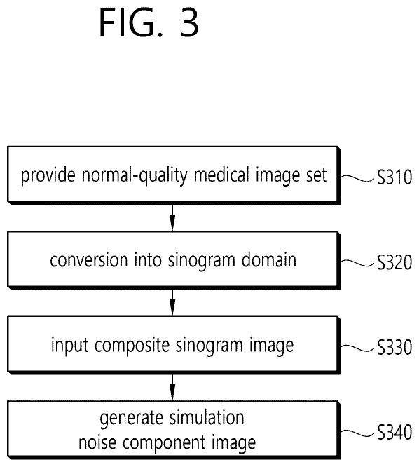

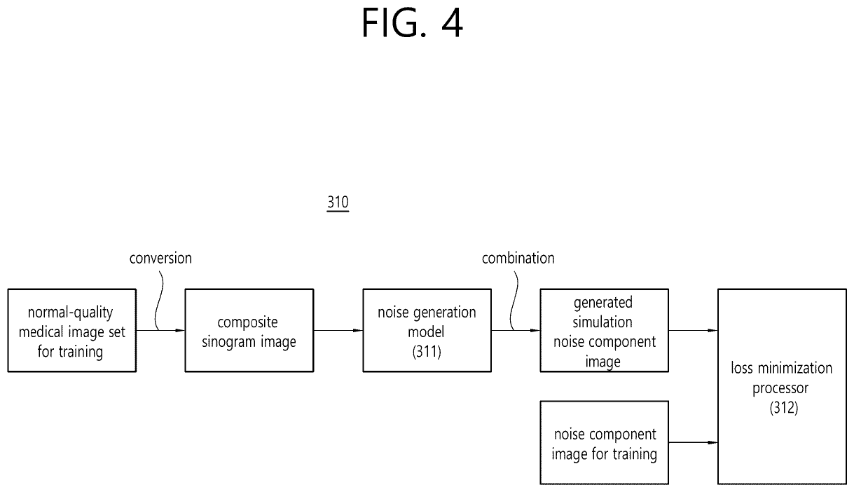

FIG. 2 is a conceptual view of generating a simulation noise component image based on a first noise simulator according to an embodiment, and FIG. 3 is a flowchart of generating the simulation noise component image based on the first noise simulator according to an embodiment. In addition, FIG. 4 is a conceptual view of showing how to train a noise model loaded into the first noise simulator according to an embodiment.

As shown in FIGS. 2 to 4, the first noise simulator 310 according to an embodiment may generate the simulation noise component image from a medical image set of normal quality.

Here, the first noise simulator may include a noise generation model 311 of which parameters are varied depending on training. The noise generation model 311 may receive a composite sinogram image generated from the medical image set of the normal quality and combine the composite sinogram image with a noise component, thereby generating the simulation noise component image.

For example, the first noise simulator 310 may receive the medical image set of the normal quality (S310), and convert the domain of the medical image set of the normal quality into a sinogram domain (S320).

For example, an attenuation coefficient for each pixel of the image, distance information between a focus of an X-ray source and a detector, and distance information between the focus of the X-ray source and a patient may be identified based on medical information about the medical image of the normal quality in the domain conversion for the medical image set of the normal quality.

Alternatively, the attenuation coefficient for each pixel may be identified by obtaining tube voltage information corresponding to the medical image of the normal quality in the domain conversion for the medical image set of the normal quality. In addition, the distance information between the focus of the X-ray source and the detector and the distance information between the focus of the X-ray source and the patient may be identified based on image information about the medical image of the normal quality.

Then, a composite sinogram is generated based on the attenuation coefficient for each pixel, the distance information between the focus of the X-ray source and the detector, and the distance information between the focus of the X-ray source and the patient so as to generate a domain conversion image of the medical image set of the normal quality. In this case, the composite sinogram image may be generated by performing image projection for each rotation angle based on the attenuation coefficient for each pixel, the distance information between the focus of the X-ray source and the detector, and the distance information between the focus of the X-ray source and the patient.

Thus, the composite sinogram image for the medical image set of the normal quality may be input to the noise generation model (S330). Thus, the noise generation model 311 combines the composite sinogram image with a noise component as trained in advance, thereby generating a noise component composite sinogram with the noise component. In addition, the noise component composite sinogram generated by the noise generation model 311 is converted into the simulation noise component image (S340). For example, the noise component composite sinogram may be filtered and back-projected to a CT image domain to generate the simulation noise component image.

Meanwhile, the noise generation model 311 may be trained based on a normal-quality medical image set for training and a noise component image set for training to generate the noise component composite sinogram.

For example, when the normal-quality medical image set for the training is subjected to the domain conversion and input to the noise generation model 311, the simulation noise component image may be generated by the noise generation model 311.

Thus, in a loss minimization processor 312 for training the noise generation model 311, the training may be performed while parameters e repeatedly adjusted to minimize a loss due to difference between the generated simulation noise component image and the noise component image for the training.

Thus, the noise generation model 311 may generate the simulation noise component image by combining the composite sinogram image for the normal-quality medical image set with the noise component. In addition, the simulation noise component image may be used in training the noise extraction deep learning model 200.

### <Second Noise Simulator>

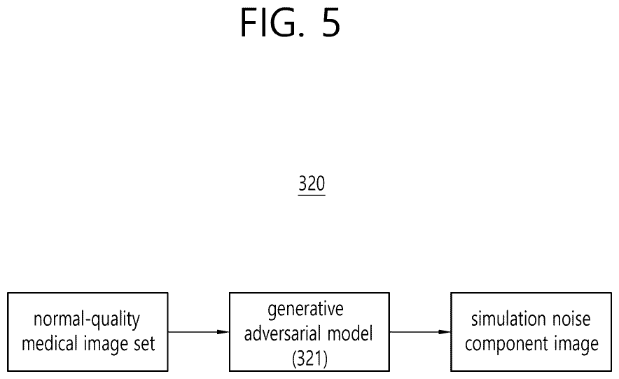

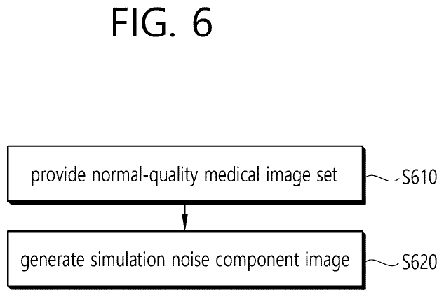

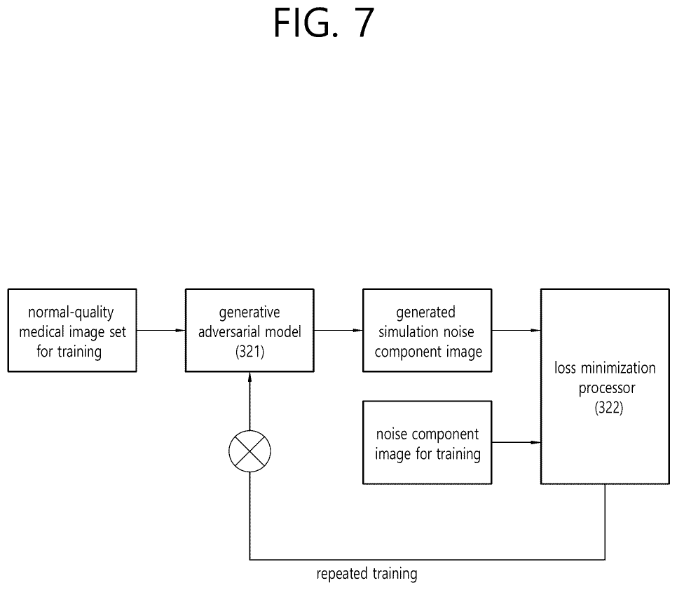

FIG. 5 is a conceptual view of generating a simulation noise component image based on a second noise simulator according to an embodiment, and FIG. 6 is a flowchart of generating the simulation noise component image based on the second noise simulator according to an embodiment. In addition, FIG. 7 is a conceptual view of showing how to train a generative adversarial model loaded into the second noise simulator according to an embodiment.

As shown in FIGS. 5 to 7, the second noise simulator 320 according to an embodiment may generate a simulation noise component image from a medical image set of normal quality.

Here, the second noise simulator 320 may include a deep learning-based generative adversarial model 321. The generative adversarial model 321 may receive the normal-quality medical image set (S610), and generate the simulation noise component image (S620). For example, the generative adversarial model 321 may include a generative adversarial network (GAN) model, but there are no limits to the types of the generative adversarial model 321.

For example, the medical image set for training, in which the normal-quality medical image and the noise component image are included forming a pair, may be used in training the generative adversarial model 321 to generate the simulation noise component image. In this case, the normal-quality medical image for the training may be input to the generative adversarial model 321, so that the generative adversarial model 321 can generate the simulation noise component image. Thus, in a loss minimization processor 322 for training the generative adversarial model 321, the training may be performed while parameters are repeatedly adjusted to minimize a loss due to difference between the generated simulation noise component image and the noise component image for the training.

Thus, the second noise simulator 320 may generate the simulation noise component image from the normal-quality medical image set.

### <Third Noise Simulator>

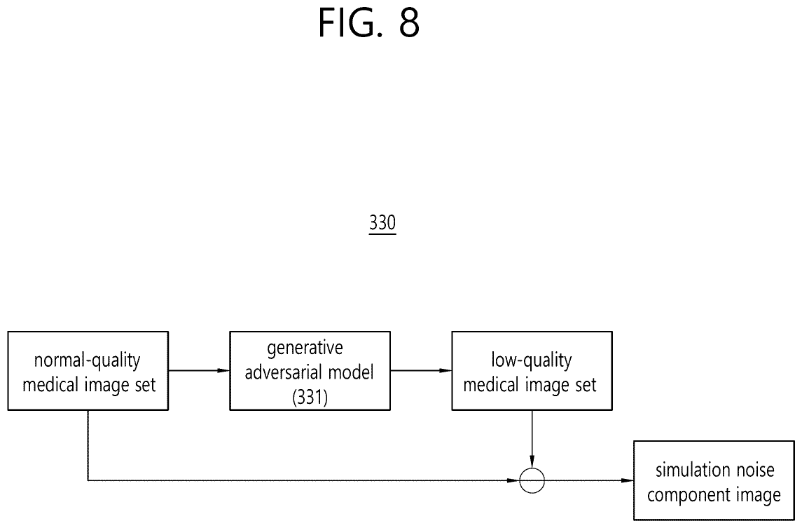

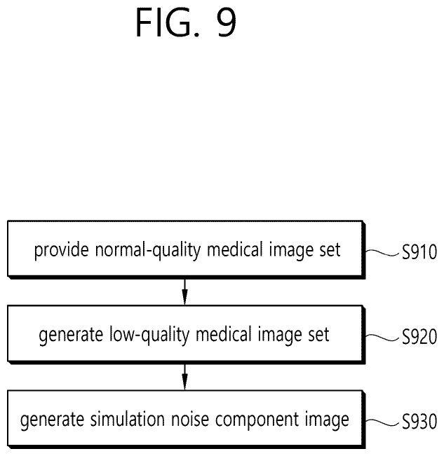

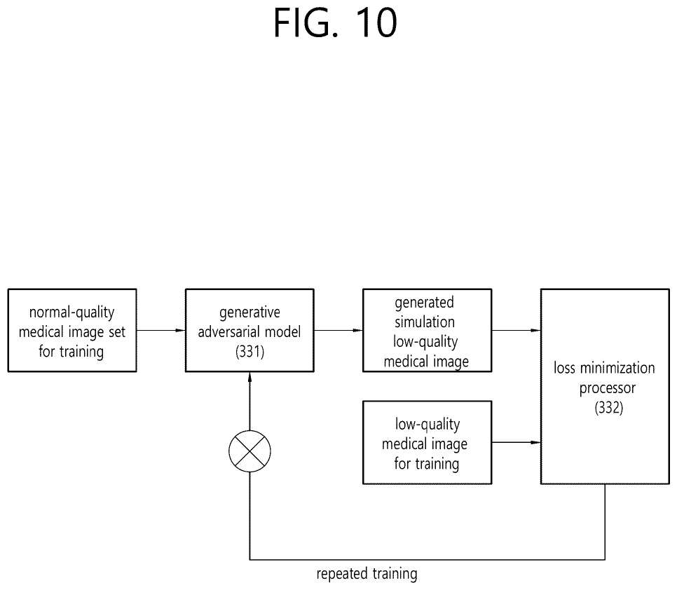

FIG. 8 is a conceptual view of generating a simulation noise component image based on a third noise simulator according to an embodiment, and FIG. 9 is a flowchart of generating the simulation noise component image based on the third noise simulator according to an embodiment. In addition, FIG. 10 is a conceptual view of showing how to train a generative adversarial model loaded into the third noise simulator according to an embodiment.

As shown in FIGS. 8 to 10, the third noise simulator 330 according to an embodiment may generate a simulation noise component image from a medical image set of normal quality.

Here, the third noise simulator 330 may include a deep learning-based generative adversarial model 331. The generative adversarial model 331 may receive the normal-quality medical image set (S910), and generate a simulation low-quality medical image generated by the generative adversarial model 331 (S920). In addition, the generated simulation low-quality medical image may be subtracted from the normal-quality medical image to generate the simulation noise component image (S930). For example, the generative adversarial model 331 may include a GAN model, but there are no limits to the types of the generative adversarial model 331.

For example, in training the generative adversarial model 331, the generative adversarial model 331 may be trained to generate the simulation low-quality medical image. In this case, the medical image set for training, in which the normal-quality medical image and the low-quality medical image are included forming a pair, may be used in training the generative adversarial model 331 to generate the simulation low-quality medical image. In this case, the normal-quality medical image for the training may be input to the generative adversarial model 331, so that the generative adversarial model 331 can generate the simulation low-quality medical image. Thus, in a loss minimization processor 332 for training the generative adversarial model 331, the training may be performed while weighted values are repeatedly adjusted to minimize a loss due to difference between the generated simulation low-quality medical image and the low-quality medical image for the training.

Thus, the third noise simulator 320 may generate the simulation noise component image by subtracting the simulation low-quality medical image generated by the generative adversarial model 321 from the normal-quality medical image.

In this way, the simulation noise component images generated from the first noise simulator 310, the second noise simulator 320, and the third noise simulator 330 may be used as data for training the noise extraction deep learning model 200.

In particular, the first noise simulator 310, the second noise simulator 320, and the third noise simulator 330 require the set of noise component images or low-quality medical images, which is paired with the normal-quality medical images, only for training the simulation noise generation model 311 and the generative adversarial models 321 and 331.

On the other hand, a large amount of training data required for training the noise extraction deep learning model 200 may be easily generated because a reference noise component image is not required when the simulation noise component image is generated through the first noise simulator 310, the second noise simulator 320, and the third noise simulator 330.

Below, a method of training the noise extraction deep learning model 200 based on a simulation noise component image I2 and a simulation low-quality image I2 generated from the simulation noise component image I2 will be described with reference to the accompanying drawings.

### <Noise Extraction Deep Learning Model>

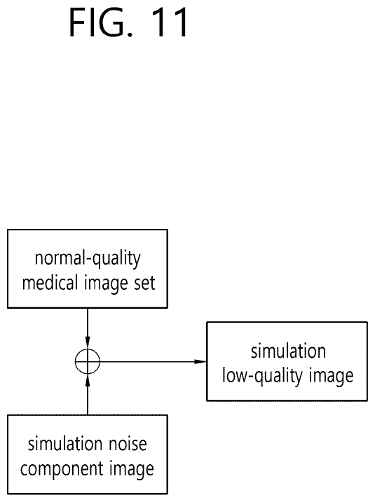

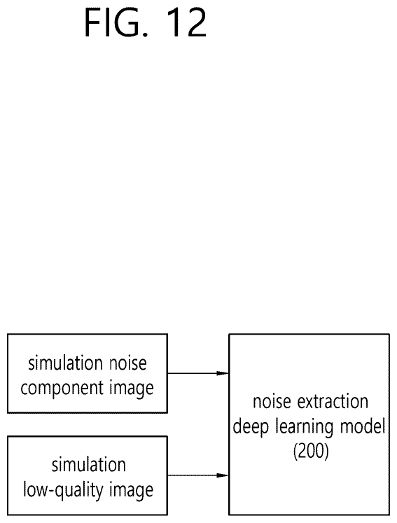

FIG. 11 is a conceptual view of generating a simulation low-quality image from a simulation noise component image according to an embodiment, and FIG. 12 is a conceptual view of training a noise extraction deep learning model according to an embodiment.

As shown in FIGS. 11 and 12, the simulation noise component image and the simulation low-quality image are used in training the noise extraction deep learning model 200 according to an embodiment.

Thus, the simulation noise component image may employ images generated by the first noise simulator 310, the second noise simulator 320, and the third noise simulator 330. In addition, the simulation low-quality image may be generated by combining the simulation noise component image and the normal-quality medical image set together.

For example, the simulation low-quality image may be generated by simply combining an image-domain simulation noise component image and an image-domain normal-quality medical image set.

Alternatively, the simulation low-quality image may be generated by converting each domain of the simulation noise component image and the normal-quality medical image into the sinogram domain, and performing combination in the sinogram domain. In addition, the composite sinogram image may be converted into the simulation low-quality image by back projection.

Then, the noise extraction deep learning model 200 is trained using the simulation low-quality image and the simulation noise component image as a pair. Thus, the noise extraction deep learning model 200 may implement the function of extracting the noise component from the medical image 11 for the processing provided by the medical apparatus 100.

Finally, the image processing module 120 may extract the noise component from the medical image 11 for the processing when receiving the medical image 11 for the processing, and subtract the noise component from the medical image 11 for the processing, thereby generating a high-resolution readout image 13.

In this way, an apparatus and method for denoising a medical image according to the disclosure can easily generate a large amount of learning data in training the noise extraction deep learning model, thereby implementing a higher performance noise extraction deep learning model.

In other words, the simulation noise component image may be advantageous for generating a large amount of learning data because the reference noise component image is not required. In addition, the simulation low-quality image may be generated by simply adding the normal-quality medical image to the simulation noise component image. Thus, many simulation low-quality images paired with many simulation noise component images are relatively easily generated, thereby improving the learning performance of the noise extraction deep learning model.

Accordingly, an apparatus and method for denoising a medical image according to the disclosure have an effect on implementing more effective noise reduction performance because a noise simulator is trained with a relatively small amount of medical image data and a larger amount of medical image data for training is generated based on the noise simulator to train a deep learning model.

Although a few embodiments of the disclosure have been described above and illustrated in the accompanying drawings, the embodiments should not be construed as limiting the technical spirit of the disclosure. The scope of the disclosure is limited only by the subject matters disclosed in the appended claims, and the technical spirit of the disclosure may be modified and changed in various forms by a person having ordinary knowledge in the art. Therefore, such modification and change obvious to those skilled in the art will fall within the scope of the disclosure.

What is claimed is:

1. An apparatus for denoising a medical image, comprising:
an image processing module configured to extract a noise component from a medical image for processing by inputting the medical image for the processing to a noise extraction deep learning model trained in advance, and generate a noise-removed image by subtracting the noise component from the medical image for the processing,
wherein the noise extraction deep learning model is trained using a simulation noise component image generated by a noise simulator and a simulation low-quality image generated based on the simulation noise component image as a pair.

2. The apparatus of claim 1, wherein the simulation low-quality image is generated by combining the simulation noise component image and a normal-quality medical image.

3. The apparatus of claim 1, wherein the noise simulator is configured to generate the simulation noise component image by inputting a set of the normal-quality medical images, of which a domain is converted into a sinogram domain, to a noise generation model trained in advance.

4. The apparatus of claim 3, wherein the noise generation model is provided as a model, of which parameters are varied depending on training, and repeatedly trained to minimize a loss due to difference between a noise component image for training and the generated simulation noise component image.

5. The apparatus of claim 1, wherein the noise simulator is configured to generate the simulation noise component image by inputting a set of the normal-quality medical images to a generative adversarial model trained in advance.

6. The apparatus of claim 5, wherein the generative adversarial model is repeatedly trained to minimize a loss due to difference between a noise component image for training and a simulation noise component image generated by inputting a set of normal-quality medical images for training to the generative adversarial model.

7. The apparatus of claim 1, wherein the noise simulator is configured to generate a set of low-quality medical images by inputting a set of normal-quality medical images to a generative adversarial model trained in advance, and generate the simulation noise component image by subtracting the set of low-quality medical images from the set of normal-quality medical images.

8. The apparatus of claim 7, wherein the generative adversarial model is repeatedly trained to minimize a loss due to difference between a low-quality medical image for training and a simulation low-quality medical image generated by inputting a set of normal-quality medical images for training to the generative adversarial model.

9. A method of denoising a medical image, comprising:
inputting a medical image for processing to a noise extraction deep learning model trained in advance;
extracting a noise component from the medical image for the processing through the noise extraction deep learning model; and
generating a noise-removed image by subtracting the noise component from the medical image for the processing,
wherein the noise extraction deep learning model is trained using a simulation noise component image generated by a noise simulator and a simulation low-quality image generated based on the simulation noise component image as a pair.

10. The method of claim 9, wherein the simulation low-quality image is generated by combining the simulation noise component image and a normal-quality medical image.

11. The method of claim 9, wherein the noise simulator is configured to generate the simulation noise component image by inputting a set of the normal-quality medical images, of which a domain is converted into a sinogram domain, to a noise generation model trained in advance.

12. The method of claim 11, wherein the noise generation model is provided as a model, of which parameters are varied depending on training, and repeatedly trained to minimize a loss due to difference between a noise component image for training and the generated simulation noise component image.

13. The method of claim 9, wherein the noise simulator is configured to generate the simulation noise component image by inputting a set of the normal-quality medical images to a generative adversarial model trained in advance.

14. The method of claim 13, wherein the generative adversarial model is repeatedly trained to minimize a loss due to difference between a noise component image for training and a simulation noise component image generated by inputting a set of normal-quality medical images for training to the generative adversarial model.

15. The method of claim 9, wherein the noise simulator is configured to generate a set of low-quality medical images by inputting a set of normal-quality medical images to a generative adversarial model trained in advance, and generate the simulation noise component image by subtracting the set of low-quality medical images from the set of normal-quality medical images.

16. The method of claim 15, wherein the generative adversarial model is repeatedly trained to minimize a loss due to difference between a low-quality medical image for training and a simulation low-quality medical image generated by inputting a set of normal-quality medical images for training to the generative adversarial model.
